(19) United States
(12) Patent Application Publication
(10) Pub. No.: US 2022/0211352 A1
(43) Pub. Date: Jul. 7, 2022

(54) SYSTEM AND METHOD FOR UTILIZING DEEP LEARNING TECHNIQUES TO ENHANCE COLOR DOPPLER SIGNALS

(71) Applicant: GE PRECISION HEALTHCARE LLC, Wauwatosa, WI (US)

(72) Inventor: Jeong Seok Kim, Milwaukee, WI (US)

(21) Appl. No.: 17/142,349

(22) Filed: Jan. 6, 2021

(51) Int. Cl. A61B 8/08; G06N 3/08

(52) U.S. Cl. CPC A61B 8/5246; G06N 3/08; A61B 8/0891

(57) ABSTRACT

A computer implemented method is provided. The method includes receiving, via a processor, a first ultrasound color Doppler image having a color Doppler signal that is inaccurate. The method also includes outputting, via the processor utilizing a generative adversarial network (GAN) system that has been trained, a second ultrasound color Doppler image based on the first ultrasound color Doppler image, wherein the second ultrasound color Doppler image accurately represents the color Doppler signal.

### BACKGROUND

The subject matter disclosed herein relates to ultrasound image processing and, more particularly, utilizing deep learning techniques to enhance ultrasound color Doppler signals.

Ultrasound color flow imaging is a Doppler technique utilized in medical diagnostics to assess the dynamics and spatial distribution of blood flow. The color Doppler signal contains blood flow information but also clutter or motion artifacts (e.g., due to the pulsation of vessel walls, heart motion, intestinal peristalsis, etc.). During signal processing, a filter (e.g., clutter filter such as a wall filter, singular value decomposition filter, etc.) to reduce the clutter may be applied to the color Doppler signals to enable obtaining high quality ultrasound color flow images. These filters include a threshold (e.g., tissue/blood threshold) or cut off based on empirical values to remove the clutter signals. However, for fine blood vessels, if the threshold is too low or small, the tissue signal may be mixed with the blood signal in the color Doppler signal and the generated image may be of poor quality due to the color Doppler signal overwhelming the displayed blood vessels (e.g., the color Doppler signal being displayed on and beyond the walls of the blood vessels as opposed to within the walls); thus, making it difficult to visualize the fine blood vessels. If the threshold is too high, the color Doppler signal may be cut off and the color Doppler signal displayed within the fine blood vessels may be difficult to visualize.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer implemented method is provided. The method includes receiving, via a processor, a first ultrasound color Doppler image having a color Doppler signal that is inaccurate. The method also includes outputting, via the processor utilizing a generative adversarial network (GAN) system that has been trained, a second ultrasound color Doppler image based on the first ultrasound color Doppler image, wherein the second ultrasound color Doppler image accurately represents the color Doppler signal.

In another embodiment, a computer implemented method is provided. The method includes training, via a processor, a generative adversarial network comprising a generator and a discriminator. Training includes providing to the generator, via the processor, a first ultrasound color Doppler image having an inaccurate color Doppler signal. Training also includes generating at the generator, via the processor, a first distribution-based image based on the first ultrasound color Doppler image. Training further includes determining at the discriminator, via the processor, whether a color Doppler signal of the first distribution-based image is accurately represented within the first distribution-based image by comparing the first distribution-based image to a second ultrasound color Doppler image having an accurate color Doppler signal. Training even further includes determining at the discriminator, via the processor, whether a color Doppler signal of the first distribution-based image is accurately represented within the first distribution-based image by comparing the first distribution-based image to a second ultrasound color Doppler image having an accurate color Doppler signal.

In a further embodiment, a generative adversarial network (GAN) system is provided. The GAN system includes a generator sub-network configured to receive a first ultrasound color Doppler image having an inaccurate color Doppler signal, wherein the generator sub-network is configured to generate a distribution-based image based on the first ultrasound color Doppler image. The GAN system also includes a discriminator sub-network configured to determine one or more loss functions indicative of errors in the distribution-based image based on a comparison of the first ultrasound color Doppler image to the second ultrasound color Doppler image having an accurate color Doppler signal. The generator sub-network is configured to be updated based on the one or more loss functions so that the generator sub-network generates subsequent distribution-based images having respective color Doppler signals that are more accurate that previous iterations of the distribution-based images.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is an embodiment of a block diagram of an ultrasound system, in accordance with aspects of the present disclosure;

FIG. 2 is an embodiment of a schematic diagram of the generation of color Doppler images from the clutter filtering of color Doppler signals;

FIG. 3 is an embodiment of a schematic diagram of a neural network architecture for use in image processing (e.g., enhancing color Doppler signals), in accordance with aspects of the present disclosure;

FIG. 4 is an embodiment of a flow chart of a method for training a generative adversarial network (GAN), in accordance with aspects of the present disclosure; and

FIG. 5 is an embodiment of a flow chart of a method for utilizing a trained GAN to enhance color Doppler signals in ultrasound color Doppler images, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

Deep-learning (DL) approaches discussed herein may be based on artificial neural networks, and may therefore encompass one or more of deep neural networks, fully connected networks, convolutional neural networks (CNNs), perceptrons, encoders-decoders, recurrent networks, wavelet filter banks, u-nets, generative adversarial networks (GANs), or other neural network architectures. The neural networks may include shortcuts, activations, batch-normalization layers, and/or other features. These techniques are referred to herein as deep-learning techniques, though this terminology may also be used specifically in reference to the use of deep neural networks, which is a neural network having a plurality of layers.

As discussed herein, deep-learning techniques (which may also be known as deep machine learning, hierarchical learning, or deep structured learning) are a branch of machine learning techniques that employ mathematical representations of data and artificial neural networks for learning and processing such representations. By way of example, deep-learning approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides for utilizing deep learning techniques to enhance color Doppler signals from fine blood vessels. In particular, a generative adversarial network (GAN) system or model is trained to receive ultrasound color Doppler images (i.e., grayscale images with superimposed color Doppler signals) of a fine blood vessel area having inaccurate color Doppler signals and to output ultrasound color Doppler images having accurate color Doppler signals. The color Doppler signals of the received ultrasound color Doppler images were filtered via a clutter filter (e.g., a singular value decomposition filter or a wall filter). Due to an empirical threshold utilized by the clutter filter, the filtered color Doppler signals may be inaccurate. For example, the color Doppler signal may have been cutoff (e.g., due to utilization of a threshold that is too large), thus, making the color Doppler signal displayed within the fine blood vessels difficult to visualize. In another scenario, the color Doppler signal may have a blood signal mixed with a tissue signal (e.g., due to utilization of a threshold that is too small) resulting in the color Doppler signal overwhelming the displayed blood vessels (i.e., blooming or color bleeding) making it difficult to visualize the fine blood vessels. The trained GAN system can improve the image quality of color Doppler images by taking an inaccurate color Doppler signal (e.g., weak color Doppler signal or color Doppler signal with blooming artifact due to mixed tissue/blood) and enhancing the color Doppler signal to generate high quality color Doppler images (i.e., equivalent to color Doppler images where an appropriate threshold was utilized during clutter filtering) with accurate color Doppler signals.

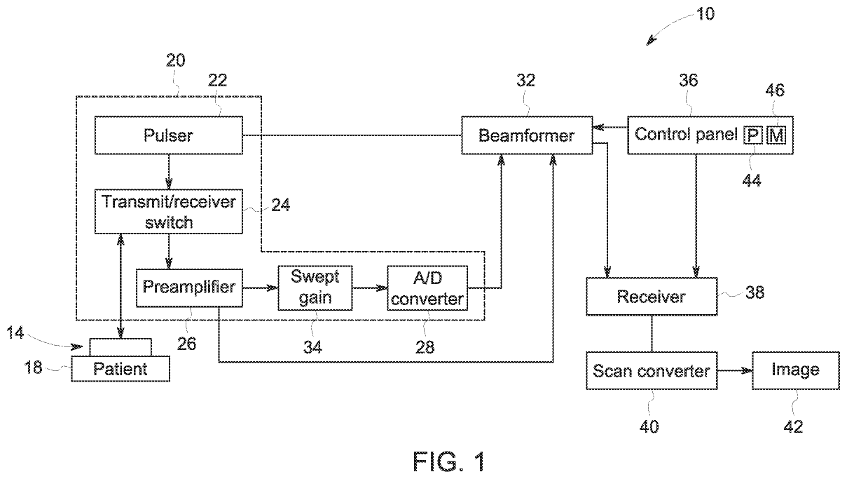

With the preceding in mind, and by way of providing useful context, FIG. 1 depicts a high-level view of components of an ultrasound system 10 that may be employed in accordance with the present approach. The illustrated ultrasound system 10 includes a transducer array 14 having transducer elements suitable for contact with a subject or patient 18 during an imaging procedure. The transducer array 14 may be configured as a two-way transducer capable of transmitting ultrasound waves into and receiving such energy from the subject or patient 18. In such an implementation, in the transmission mode the transducer array elements convert electrical energy into ultrasound waves and transmit it into the patient 18. In reception mode, the transducer array elements convert the ultrasound energy received from the patient 18 (backscattered waves) into electrical signals.

Each transducer element is associated with respective transducer circuitry, which may be provided as one or more application specific integrated circuits (ASICs) 20, which may be present in a probe or probe handle. That is, each transducer element in the array 14 is electrically connected to a respective pulser 22, transmit/receive switch 24, preamplifier 26, swept gain 34, and/or analog to digital (A/D) converter 28 provided as part of or on an ASIC 20. In other implementations, this arrangement may be simplified or otherwise changed. For example, components shown in the circuitry 20 may be provided upstream or downstream of the depicted arrangement, however, the basic functionality depicted will typically still be provided for each transducer element. In the depicted example, the referenced circuit functions are conceptualized as being implemented on a single ASIC 20 (denoted by dashed line), however it may be appreciated that some or all of these functions may be provided on the same or different integrated circuits.

Also depicted in FIG. 1, a variety of other imaging components are provided to enable image formation with the ultrasound system 10. Specifically, the depicted example of an ultrasound system 10 also includes a beam former 32, a control panel 36, a receiver 38, and a scan converter 40 that cooperate with the transducer circuitry to produce an image or series of images 42 that may be stored and/or displayed to an operator or otherwise processed as discussed herein. A processing component 44 (e.g., a microprocessor) and a memory 46 of the system 10, such as may be present control panel 36, may be used to execute stored routines for processing the acquired ultrasound signals to generate meaningful images and/or motion frames (including color Doppler images with color Doppler signals superimposed on grayscale images), which may be displayed on a monitor of the ultrasound system 10. The processing component 44 may also filter (e.g., clutter filter) the color Doppler signals utilizing a single value decomposition filter or a wall filter. The processing component 44 may further utilize a generative adversarial network (GAN) system or model stored on the memory 46 to generate ultrasound color Doppler images with enhanced color Doppler signals (e.g., improved image quality) from ultrasound color Doppler images having color Doppler signals that are inaccurate (e.g., of poor image quality).

In a present embodiment, the ultrasound system 10 is capable of acquiring one or more types of volumetric flow information within a vessel or vessels (e.g., fine blood vessels). That is, the plurality of reflected ultrasound signals received by the transducer array 14 are processed to derive a spatial representation that describes one or more flow characteristics of blood within the imaged vasculature. For example, in one embodiment, the ultrasound system 10 is suitable for deriving spectral or color-flow type Doppler information pertaining to one or more aspects of blood flow or velocity within the region undergoing imaging (e.g., color Doppler or color flow Doppler velocity information for planar or volume flow estimation). Similarly, various volumetric flow algorithms may be used to process or integrate acquired ultrasound data to generate volumetric flow information corresponding to the sample space inside a blood vessel.

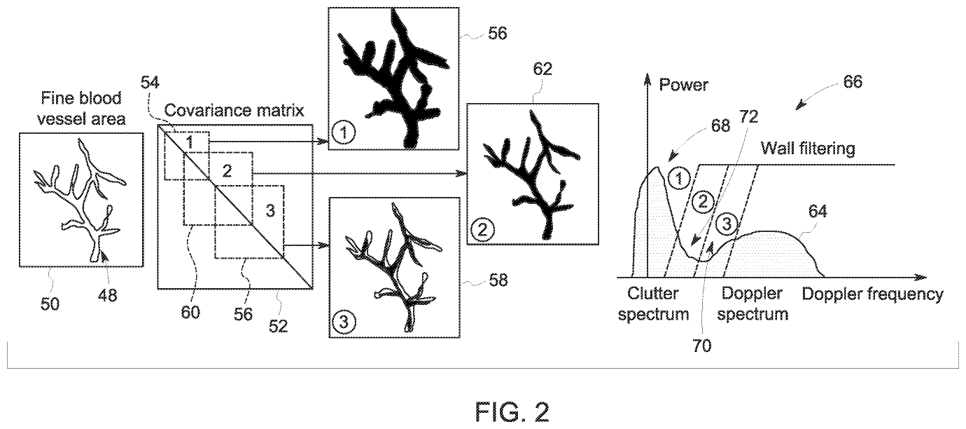

FIG. 2 is an embodiment of a schematic diagram of the generation of color Doppler images from clutter filtering of color Doppler signals. As depicted, a fine blood vessel area 48 (as depicted in grayscale image 50) may be subjected to ultrasound color flow imaging utilizing the ultrasound system 10 described in FIG. 1. A filter (e.g., clutter filter) may be applied to color Doppler signal to reduce clutter or motion artifacts (e.g., due to the pulsation of vessel walls, heart motion, intestinal peristalsis, etc.). The filter may be a singular value decomposition (SVD) filter that separates the blood signal from tissue clutter and noise based on different characteristics of different components of the signal when projected onto a singular value domain. For example, a covariance matrix 52 of the color Doppler signal is subject to thresholding (e.g., one or more empirical thresholds) to remove a certain number of singular vectors from the color Doppler signal. If the threshold is too small, the color Doppler signal data utilized 54 (labeled 1 on the covariance matrix 52) may include the blood signal being mixed with a tissue signal resulting in the color Doppler signal overwhelming the displayed blood vessels (i.e., blooming or color bleeding) making it difficult to visualize the fine blood vessels as illustrated in the ultrasound color Doppler image 56. If the threshold too big, the color Doppler signal data utilized 56 (labeled 3 on the covariance matrix 52) may cut off the blood signal and the color Doppler signal displayed within the fine blood vessels 48 may be difficult to visualize as illustrated in the ultrasound color Doppler image 58. If the color Doppler signal data utilized 60 (labeled 2 on the covariance matrix 52) is between the low and high thresholds, the color Doppler signal obtained more accurately reflects the blood flow information as indicated in the ultrasound Doppler image 62.

Alternatively, the filter may be a wall filter (e.g., high pass filter) that separates the blood signal from the tissue clutter and noise. In utilizing the wall filter, the color Doppler signal is subjected to thresholding (e.g., one or more empirical thresholds). The wall filter may remove low and/or high frequency portions of the color Doppler signal. The application of wall filtering to a color Doppler signal 64 is illustrated in graph 66. Similar to the SVD filter, if the threshold is too small, the color Doppler signal data utilized 68 (labeled 1 on the graph 66) may include the blood signal being mixed with a tissue signal resulting in the color Doppler signal overwhelming the displayed blood vessels (i.e., blooming or color bleeding) making it difficult to visualize the fine blood vessels as illustrated in the ultrasound color Doppler image 56. If the threshold too big, the color Doppler signal data utilized 70 (labeled 3 on the graph 66) may cut off the blood signal and the color Doppler signal displayed within the fine blood vessels 48 may be difficult to visualize as illustrated in the ultrasound color Doppler image 58. If the color Doppler signal data utilized 72 (labeled 2 on the graph 66) is between the low and high thresholds, the color Doppler signal obtained more accurately reflects the blood flow information as indicated in the ultrasound Doppler image 62. The inaccurate ultrasound color Doppler images 56 and 58 although having inaccurate color Doppler signals still include valuable blood flow information that may be resolved utilizing the deep learning techniques described herein.

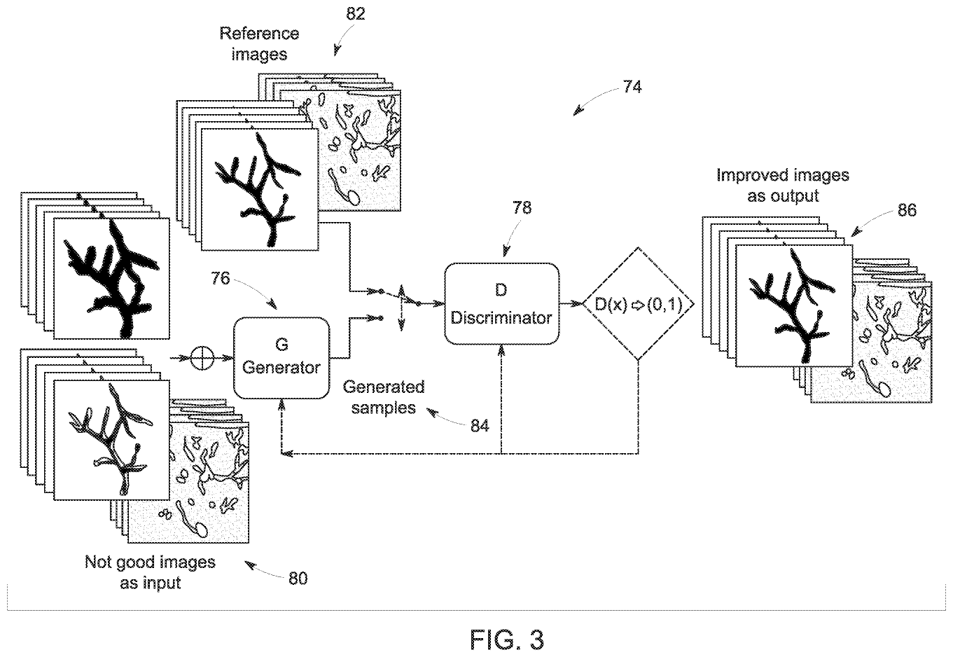

FIG. 3 is a schematic diagram of the neural network architecture of a GAN system or model 74 for use in enhancing color Doppler signals from fine blood vessels. The GAN 74 includes a generator or generator sub-network or model 76 (e.g., de-convolutional neural network) and a discriminator or discriminator sub-network or model 78 (e.g., convolutional neural network). The generator 76 is trained to produce improved (in image quality due to an enhanced color Doppler signal) ultrasound color Doppler images with accurate color Doppler signals from ultrasound color Doppler signals with inaccurate color Doppler signals (e.g., due to blooming or a cutoff signal). The discriminator 78 distinguishes between real data (e.g., from ultrasound color Doppler images having accurate color Doppler signals) and generated data (generated by the generator 76). In addition, the discriminator 78 enables the generator 76 to generate more realistic information from the learned data distribution.

The GAN 74 may receive color Doppler images of poor quality 80 (e.g., having inaccurate color Doppler signals similar to the images 56, 58 in FIG. 2). The color Doppler signals in these poor quality ultrasound color Doppler images 80 were subjected to clutter filtering (e.g., wall filtering or SVD filtering). These poor quality ultrasound color Doppler images 80 are provided to the generator 76 as an input. The generator 76 generates samples or distribution-based images 84 from these poor quality ultrasound color Doppler images 80. The GAN 74 also receives reference images 82 (e.g., ultrasound color Doppler images having accurate color Doppler signals) that are provided to the discriminator 78 for comparison by the discriminator 78 to the reference images 82. In particular, the discriminator 78 maps the generated images (i.e., distribution-based images 84) to a real data distribution D: D(xᵢ) [0, 1] derived from the reference images 82. The generator 76 learns to map the representations of latent space to a space of data distribution G→ [symbol] ^{|x|,} where z∈ [symbol] ^{|x|} represents the samples from the latent space x∈ [symbol] ^{|x|} of image distribution. The generator 76 is configured to learn the distribution p_{θ}(x), approximate to the real distribution pᵣ(x) derived from the reference images 82, and to generate samples p_{G}(x) (i.e., the distribution-based images 84) where the probability distribution function of the generated samples p_{G} (x) equals the probability density function of the real samples pᵣ(x). This can be achieved by learning directly and optimizing through maximum likelihood the differential function p_{θ}(x) so that that p_{θ}(x)>0 and ƒₓ p_{θ}(x)dx=1. Alternatively, the differential transformation function q_{θ}(z) of p_{θ}(x) can be learned and optimized through maximum likelihood where z is the existing common distribution (e.g., uniform or Gaussian distribution).

The discriminator 78 has to recognize the data from the real data distribution pᵣ(x), where D indicates the estimated probability of data points xᵢ∈ [symbol] ⁿ. In case of binary classification, if the estimated probability D(xᵢ): -> [symbol] n[0, 1] is the positive class pᵢ and 1-D(xᵢ) [0, 1] is the negative class qᵢ, the cross entropy distribution between pᵢ and qᵢ is, L(p, q)=−Σᵢⁿpᵢ log qᵢ. For a given point xᵢ and corresponding label yᵢ, the data distribution xᵢ can be from the real data xᵢ˜pᵣ(x) (e.g., from the reference images 82) or the generator data xᵢ˜p_{g} (z) (e.g., from the distribution-based images 84). Considering exactly half of data from the two sources such as real, fake, the generator 76 and discriminator 78 tend to fight each other in a minmax game to minimize the loss function. The loss function is as follows:

$$\begin{matrix}{min}_{G} {max}_{D} L ( {( {x}_{i} , {y}_{i} )}_{i = 1}^{n} , D ) = - \frac{1}{2} {E}_{x ∼ {p}_{r} ( x )} log D ( x ) - \frac{1}{2} {E}_{z ∼ {p}_{r} ( z )} log [ 1 - D ( G ( z ) ) ] + λΨ \\ or & ( 1 ) \\ {min}_{G} {max}_{D} L ( G , D ) = - \frac{1}{2} {E}_{x ∼ {p}_{r} ( x )} log D ( x ) - \frac{1}{2} {E}_{z ∼ {p}_{r} ( z )} log [ 1 - D ( G ( z ) ) ] + λΨ , & ( 2 )\end{matrix}$$

where λΨ=E_{x˜p}_{ᵣ}₍ₓ_{^{˜}}₎[(∥□ₓ_{^{˜}}∥²−1)²] is a term that enables overcoming the gradient vanish effect.

The loss function, which is indicative of errors is fed back (via back propagation) to the generator 76 and/or the discriminator 78. This enables the generator 76 to become further trained and once trained enough to generate distribution-based images 84 (derived from the poor quality color Doppler images 80) that may fool the discriminator 78 and be outputted by the GAN 74 as ultrasound color Doppler images 86 having accurate color Doppler signals. The trained GAN 74 will provide higher quality images to practitioners in diagnosing patients.

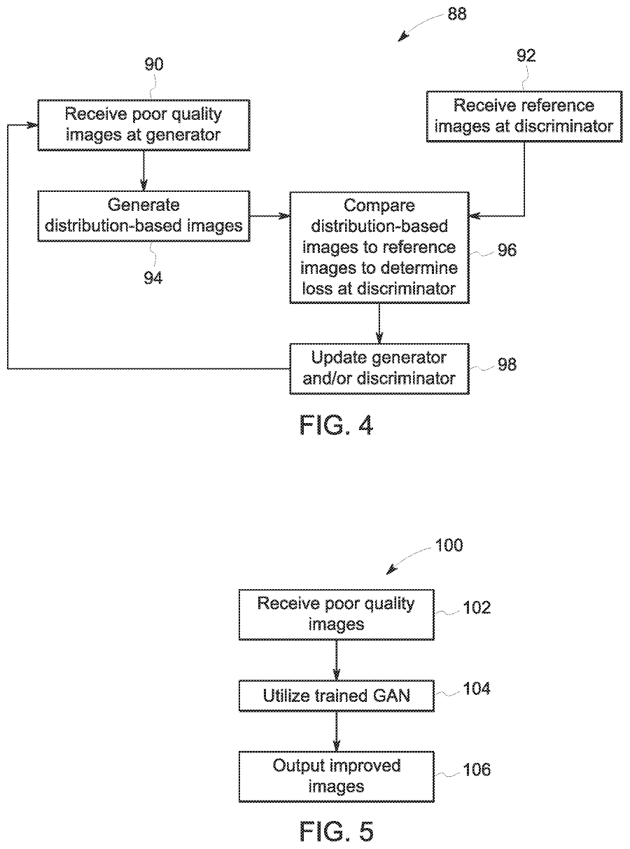

FIG. 4 is an embodiment of a flow chart of a method 88 for training a generative adversarial network (GAN) (e.g., GAN 74 in FIG. 3), in accordance with aspects of the present disclosure. The method 88 may be performed by the control panel 36 of the ultrasound system 10 in FIG. 1 or a remote processing device. The method 88 includes receiving one or more poor quality ultrasound color Doppler images at a generator of a GAN (block 90). The poor quality ultrasound color Doppler images are ultrasound color Doppler signals with inaccurate color Doppler signals (e.g., due to blooming or a cutoff signal). In addition, the color Doppler signals of the poor quality ultrasound color Doppler images were subjected to clutter filtering (e.g., wall filtering or SVD filtering). The method 88 also includes receiving one or more reference images at the GAN (block 92). The references images are ultrasound color Doppler images having accurate color Doppler signals. In addition, the color Doppler signals of the reference images were subjected to clutter filtering (e.g., wall filtering or SVD filtering).

The method 88 further includes generating one or more distribution based images (i.e., ultrasound color Doppler images) based on the poor quality ultrasound color Doppler images (block 94). The method 88 includes comparing the distribution-based images to the reference images to determine whether the respective color Doppler signals are accurately represented within the distribution-based images (block 96). In particular, the comparison includes the discriminator determining one or more loss functions indicative of errors based on the comparison between the distribution-based images and the reference images. The method 88 includes updating the generator and/or discriminator based on the comparison between the distribution-based images and the reference images (block 98). In particular, the generator and/or discriminator is updated based on the one or more loss functions. Updating the generator based on the loss functions enables the generator to generate subsequent distribution-based images having respective color Doppler signals that are more accurate than the color Doppler signals of earlier iterations of distribution-based images. These steps in the method 88 repeat until the generator is trained to generate distribution-based images where the loss functions are minimal enough that the discriminator cannot distinguish the distribution-based images from the reference images.

FIG. 5 is an embodiment of a flow chart of a method 100 for utilizing a trained GAN to enhance color Doppler signals in ultrasound color Doppler images, in accordance with aspects of the present disclosure. The method 100 may be performed by the control panel 36 of the ultrasound system 10 in FIG. 1 or a remote processing device. The method 100 includes receiving one or more poor quality ultrasound color Doppler images (e.g., as input to the generator of a GAN) (block 102). The poor quality ultrasound color Doppler images are ultrasound color Doppler signals with inaccurate color Doppler signals (e.g., due to blooming or a cutoff signal). In addition, the color Doppler signals of the poor quality ultrasound color Doppler images were subjected to clutter filtering (e.g., wall filtering or SVD filtering). The method 100 also includes utilizing a trained GAN on the poor quality ultrasound color Doppler images to generate improved quality ultrasound color Doppler images (e.g., having accurate color Doppler signals) based on the poor quality ultrasound color Doppler images (block 104). The method 100 further includes outputting the improved quality ultrasound color Doppler images (e.g., having accurate color Doppler signals) from the GAN (block 106).

Technical effects of the disclosed embodiments include utilizing deep learning techniques to enhance color Doppler signals from fine blood vessels. In particular, a generative adversarial network (GAN) system or model is trained to receive ultrasound color Doppler images (i.e., grayscale images with superimposed color Doppler signals) of a fine blood vessel area having inaccurate color Doppler signals and to output ultrasound color Doppler images having accurate color Doppler signals. The techniques provide a way to process poor quality color ultrasound Doppler images to generate improved quality color ultrasound Doppler images (e.g., having more accurate or enhanced color Doppler signals) to assist practitioners in diagnosing patients.

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer implemented method, comprising:
receiving, via a processor, a first ultrasound color Doppler image having a color Doppler signal that is inaccurate; and
outputting, via the processor utilizing a generative adversarial network (GAN) system that has been trained, a second ultrasound color Doppler image based on the first ultrasound color Doppler image, wherein the second ultrasound color Doppler image accurately represents the color Doppler signal.

2. The computer implemented method of claim 1, wherein the first ultrasound color Doppler image is of a fine blood vessel area.

3. The computer implemented method of claim 1, wherein the color Doppler signal comprises a clutter filtered color Doppler signal.

4. The computer implemented method of claim 3, wherein the clutter filtered color Doppler signal was generated via a singular value decomposition filter or a wall filter applied to the color Doppler signal.

5. The computer implemented method of claim 1, wherein the GAN system comprises a generator and a discriminator, and the method comprises training the GAN system by:
providing to the generator, via the processor, one or more ultrasound color Doppler images having respective color Doppler signals that are inaccurate;
generating at the generator, via the processor, one or more distribution-based images based on the one or more ultrasound color Doppler images having respective color Doppler signals that are inaccurate;
determining at the discriminator, via the processor, whether the respective color Doppler signals of the one or more distribution-based images are accurately represented within the one or more distribution-based images by comparing the distribution-based images to one or more ultrasound color Doppler images having respective color Doppler signals that are accurate; and
updating the generator, via the processor, based on the comparison of the one or more distribution-based images to the one or more ultrasound color Doppler images having respective color Doppler signals that are accurate.

6. The computer implemented based method of claim 5, comprising determining at the discriminator, via the processor, one or more loss functions indicative of errors in the one or more distribution-based images based on the comparison to the one or more ultrasound color Doppler images having respective color Doppler signals that are accurate.

7. The computer implemented method of claim 6, wherein updating the generator, via the processor, comprises updating the generator based on the one or more loss functions so that the generator generates subsequent distribution-based images having respective color Doppler signals that are more accurate.

8. A computer implemented method, comprising:
training, via a processor, a generative adversarial network (GAN) system comprising a generator and a discriminator by:
providing to the generator, via the processor, a first ultrasound color Doppler image having an inaccurate color Doppler signal;
generating at the generator, via the processor, a first distribution-based image based on the first ultrasound color Doppler image;
determining at the discriminator, via the processor, whether a color Doppler signal of the first distribution-based image is accurately represented within the first distribution-based image by comparing the first distribution-based image to a second ultrasound color Doppler image having an accurate color Doppler signal; and
updating the generator, via the processor, based on the comparison of the first distribution-based image to the second ultrasound color Doppler image.

9. The computer implemented method of claim 8, comprising determining at the discriminator, via the processor, one or more loss functions indicative of errors in the first distribution-based image based on the comparison to the second ultrasound color Doppler image.

10. The computer implemented method of claim 9, wherein updating the generator, via the processor, comprises updating the generator based on the one or more loss functions so that the generator generates subsequent distribution-based images having respective color Doppler signals that are more accurate that previous iterations of the distribution-based images.

11. The computer implemented method of claim 8, comprising:
providing to the generator, via the processor, a third ultrasound color Doppler image having an inaccurate color Doppler signal; and
generating at the generator, via the processor, a second distribution-based image based on the third ultrasound color Doppler image having a more accurate color Doppler signal than the first distribution-based image.

12. The computer implemented method of claim 8, comprising utilizing a trained GAN system to:
receive, via the processor, a third ultrasound color Doppler image having a color Doppler signal that is inaccurate; and
output, via the processor, a fourth ultrasound color Doppler image based on the third ultrasound color Doppler image, wherein the forth ultrasound color Doppler image accurately represents the color Doppler signal.

13. The computer implemented method of claim 8, wherein the first and second ultrasound color Doppler images are of a fine blood vessel area.

14. The computer implemented method of claim 8, wherein the inaccurate color Doppler signal of the first ultrasound color Doppler image and the accurate color Doppler signal of the second ultrasound color Doppler image comprise clutter filtered color Doppler signals.

15. The computer implemented method of claim 14, wherein the clutter filtered color Doppler signals were generated via a singular value decomposition filter or a wall filter applied to the inaccurate color Doppler signal of the first ultrasound color Doppler image and the accurate color Doppler signal of the second ultrasound color Doppler image.

16. A generative adversarial network (GAN) system, comprising:
a generator sub-network configured to receive a first ultrasound color Doppler image having an inaccurate color Doppler signal, wherein the generator sub-network is configured to generate a distribution-based image based on the first ultrasound color Doppler image; and
a discriminator sub-network configured to determine one or more loss functions indicative of errors in the distribution-based image based on a comparison of the first ultrasound color Doppler image to the second ultrasound color Doppler image having an accurate color Doppler signal, wherein the generator sub-network is configured to be updated based on the one or more loss functions so that the generator sub-network generates subsequent distribution-based images having respective color Doppler signals that are more accurate that previous iterations of the distribution-based images.

17. The GAN system of claim 16, wherein the GAN system is configured upon training to receive a third ultrasound color Doppler image having a color Doppler signal that is inaccurate and output a fourth ultrasound color Doppler image based on the third ultrasound color Doppler image, wherein the forth ultrasound color Doppler image accurately represents the color Doppler signal.

18. The GAN system of claim 16, wherein the first and second ultrasound color Doppler images are of a fine blood vessel area.

19. The GAN system of claim 18, wherein the inaccurate color Doppler signal of the first ultrasound color Doppler image and the accurate color Doppler signal of the second ultrasound color Doppler image comprise clutter filtered color Doppler signals.

20. The GAN system of claim 19, wherein the clutter filtered color Doppler signals were generated via a singular value decomposition filter or a wall filter applied to the inaccurate color Doppler signal of the first ultrasound color Doppler image and the accurate color Doppler signal of the second ultrasound color Doppler image.
